(19) United States
(12) Patent Application Publication
(10) Pub. No.: US 2024/0200068 A1
(43) Pub. Date: Jun. 20, 2024

(54) RNAi-NANOPARTICLE CONJUGATE, COMPOSITION, AND METHOD OF SYNTHESIS THEREOF

(71) Applicant: Council of Scientific & Industrial Research, New Delhi (IN)

(72) Inventors: Rathna Venkata Naga Gundloori, Pune (IN); Lekha Dinesh Kumar, Hyderabad (IN); Amarnath Ram Singam, Pune (IN)

(21) Appl. No.: 18/340,588

(22) Filed: Jun. 23, 2023

Related U.S. Application Data
(60) Provisional application No. 63/436,802, filed on Jan. 3, 2023.

(30) Foreign Application Priority Data
Dec. 15, 2022 (IN) 202211072553

(51) Int. Cl. C12N 15/113; A61P 35/00

(52) U.S. Cl. CPC C12N 15/113; A61P 35/00; C12N 2310/11; C12N 2310/122

(57) ABSTRACT

RNAi nanoparticle conjugates and compositions thereof for management of cancer include an active ingredient, a polysaccharide, an RNAi silencer, and a coating agent. The conjugates are synthesized by encapsulating an active ingredient in a polysaccharide to obtain a nanoencapsulate that is fabricated into nanoparticles. The nanoencapsulate is immobilized with an RNAi silences to obtain a complex that is coated to form the conjugate. The conjugate is non-toxic, silences EphB4 gene, and is capable of targeted cancer therapeutics with enhanced permeation. The conjugate synergistically upregulates Wnt genes by RNAi and demonstrates tumor suppressive action of the active ingredient.

### CROSS REFERENCES TO RELATED APPLICATIONS

The present application claims benefit of priority under 35 U.S.C. § 119(a)-(d) to Indian Patent Application No. 202211072553, filed Dec. 15, 2022, and under 35 U.S.C. § 119(e) to U.S. Provisional Application No. 63/436,802, filed Jan. 3, 2023. The entire contents of both priority applications are incorporated herein by reference.

### TECHNICAL FIELD

The present disclosure relates generally to pharmaceuticals. More specifically, the disclosure is directed to an RNAi nanoparticle (NP) conjugate for management of cancer. The disclosure also provides a pharmaceutical composition comprising the conjugate and a method of synthesis of the conjugate.

### BACKGROUND

The following background description includes information that may be useful for understanding. It is not an admission that any of the information provided herein is prior art or relevant to the claims presented herein, or that any publication specifically or implicitly referenced is prior art.

Cancer is a leading cause of death worldwide second only to cardiovascular diseases. Nearly 10 million deaths in the year 2020 were caused by cancer. Although different treatment modalities are available for cancer most of them are associated with debilitating side effects and lower patient survival. Moreover, targeted drug delivery poses a great challenge, especially in cancers such as colon cancer due to the site of origin of primary tumor.

Most of the existing chemotherapeutic agents are directed towards tyrosine kinases to suppress cancer. One of the significant tyrosine kinase receptors overexpressed in various cancers is the Ephb4 receptor, which is associated with tumor angiogenesis, growth, and metastasis (Cancer Res., 2007, 67, 9800-9808). Accumulating evidence has shown that tyrosine kinase Ephb4 is overexpressed in various cancers and various background experiments have specifically demonstrated that shutting down Ephb4 gene using its shRNA not only controlled the upregulated Ephb4 but also other oncogenes in the Wnt signalling pathway which is primarily responsible for colon cancer.

With the advent of next generation technologies like RNA interference (RNAi) and nanotechnology, innovative and personalized biological anticancer treatment can be expected to find its way from bench to bedside. RNAi, a post-transcriptional gene silencing phenomenon, is gaining immense clinical attention regarding its usage as a potential weapon against solid tumors. The lack of safe and effective delivery methods for RNAi molecules remains the primary challenge that prevents the full utilization of the potential of RNAi-based therapy in biological systems (D. Hattab, A. M. Gazzali and A. Bakhtiar, Pharmaceutics, 2021, 13(7), 1009) while existing nanotechnologies may be limited by its non-selective targeting.

The inherent limitations of existing therapeutic strategies, along with current treatment impediments, accentuate the need for more specific and targeted alternative cancer therapies. In spite of the current clinical management being dependent on applying robust pathological variables and well-defined therapeutic strategies; there remains an imminent need in the art for novel and targeted therapies.

Yet another object of the present disclosure is to provide a method of synthesizing an RNAi-nanoparticle conjugate.

### SUMMARY

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

Aspects of the present disclosure provide an RNAi-nanoparticle conjugate or biodrug as a feasible, biosmart, and effective drug delivery system.

In an aspect, the present disclosure provides an RNAi-nanoparticle conjugate comprising a hydrophobic active ingredient; a polysaccharide; an RNA interference (RNAi) silencer EphB4-shRNA; and a coating agent; wherein the active ingredient is encapsulated by the polysaccharide to give a nanoencapsulate(Nep); the RNAi silencer is immobilized on the Nep and coated by the coating agent.

In an embodiment, the active ingredient may be any well-known chemical, biological or naturally occurring ingredient, including but not limited to, anti-cancer ingredients selected from curcumin, resveratrol, vincristine, luteolin, quercetin, piperine, berberine; monoclonal antibodies including siltuximab, tositumomab, or herceptin; anthracyclines including doxorubicin, daunorubicin, bleomycin, orepirubicin; sorafenib, erlotinib, cisplatin, oxaliplatin, carboplatin, or combinations thereof.

In an embodiment, the RNA interference (RNAi) silencer EphB4-shRNA is EphB4-shRNA-800.

In an embodiment, the polysaccharide may be selected from, but is not limited to, chitosan, ethyl cellulose, carboxymethyl cellulose, hydroxyl propylmethyl cellulose, methylcellulose, ethyl cellulose, pectin, carrageenan, hyaluronic acid, guar gum, sodium alginate, or combinations thereof.

In an embodiment, the ratio of the active ingredient to polysaccharide may be about 1:1 to about 1:5. In another embodiment, the ratio of Nep to RNAi silencer EphB4-shRNA is in the range of about 25:0.1 to about 25:1.

In an embodiment, the coating agent may be any well-known coating agent selected from, but not limited to, polymethacrylate based copolymers including methyl acrylate, methyl methacrylate, methacrylic acid, [2-(dimethylamino)ethyl methacrylate], 3- or hydroxyethylmethacrylate; cellulose derivatives including cellulose acetate, hydroxypropyl methylcellulose, or hydroxypropyl cellulose; or combinations thereof.

In an embodiment, the conjugate is nanosized with a size in a range of about 50 nm to 500 nm.

In a preferred embodiment, the present disclosure provides an RNAi-nanoparticle conjugate comprising curcumin; chitosan; an RNA interference (RNAi) silencer EphB4-shRNA; and a polymethacrylate based copolymer; wherein the curcumin is encapsulated by chitosan to give a Nep; the RNAi silencer is immobilized on the Nep and coated by the polymethacrylate based copolymer.

In an aspect, the present disclosure provides use of an RNAi-nanoparticle conjugate for management of cancer.

In an aspect, the present disclosure provides a pharmaceutical composition comprising an RNAi-nanoparticle conjugate comprising a hydrophobic active ingredient; a polysaccharide; an RNA interference (RNAi) silencer EphB4-shRNA; and a coating agent; wherein the active ingredient is encapsulated by the polysaccharide to give a Nep; the RNAi silencer is immobilized on the Nep and coated by the coating agent; and one or more pharmaceutically active excipient(s).

In another aspect, the present disclosure provides a method for synthesis of an RNAi-nanoparticle conjugate, the method comprising the steps of: (a) fabricating a Nep comprising a hydrophobic active ingredient encapsulated by a polysaccharide; (b) complexing the Nep with an RNA interference (RNAi) silencer EphB4-shRNA at a temperature in a range of about 40-60° C.; and (c) coating with a coating agent to give the conjugate.

In an embodiment, the method comprises the steps of: (a) dispersing the active ingredient in the polysaccharide; (b) fabricating the Nep from the dispersion; (c) complexing the Nep with the RNA interference (RNAi) silencer EphB4-shRNA at a temperature in a range of about 40° C. to 60° C.; and (d) coating with the coating agent.

In a preferred embodiment, the method comprises the steps of: (a) dispersing curcumin in chitosan; (b) fabricating the Nep from the dispersion by electrospraying; (c) complexing the Nep with the RNA interference (RNAi) silencer EphB4-shRNA at a temperature in a range of about 40° C. to 60° C.; and (d) coating with polymethacrylate based copolymer.

In an aspect, the present disclosure provides a method of silencing Ephb4 receptor in a subject by administering an effective amount of the RNAi-nanoparticle conjugate or the pharmaceutical composition as recited above.

In an aspect, the present disclosure provides a method of management or treatment of cancer in a subject by administering an effective amount of the RNAi-nanoparticle conjugate or the pharmaceutical composition as recited above.

An object of the present disclosure is to provide an RNAi-nanoparticle conjugate with targeted therapeutics and enhanced permeability and retention.

An object of the present disclosure is to provide an RNAi-nanoparticle conjugate capable of silencing Ephb4 receptor for anti-cancer activity.

Another object of the present disclosure is to provide a pharmaceutical composition comprising RNAi-nanoparticle conjugate.

Other aspects will be set forth in the description that follows, and in part will be apparent from the description, or may be learned by the practice of embodiments of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further illustrate aspects of the present disclosure. The disclosure may be better understood by reference to the drawings in combination with the detailed description of the specific embodiments presented herein.

FIG. 1A-FIG. 1H depict characterizations of nanoparticles (NPs) and conjugates, as per an exemplary embodiment of the present disclosure.

FIG. 1A is an SEM micrograph of the conjugate, with a TEM image of the conjugate in the upper left corner.

FIG. 1B is an SEM image of the conjugate.

FIG. 1C is a frequency distribution graph of the conjugates (median size 123±9 nm).

FIG. 1D is a graph of Zeta potential charge measurements of curcumin (CU) NPs; curcumin-chitosan (CU-CS) Nep; curcumin-chitosan-RNAi NPs (CU-CS-RNAi) and the conjugate (CU-CS-RNAi-ES)(ES-Eudragit S-100).

FIG. 1E includes FTIR spectra of CU NPs, Nep (CU-CS), and CU coated with CS and ES NPs (CU-CS-ES).

FIG. 1F is a differential scanning colorimetric analysis of CU, CS, ES and CU-CS-ES NPs.

FIG. 1G is a graph of cumulative percentage curcumin release (%) at pH 1.2 from CU-CS Nep and CU-CS-ES NPs.

FIG. 1H is a graph of cumulative percentage curcumin release (%) at pH of 6.8 and 7.4 from CU-CS Nep and CU-CS-ES NPs.

FIG. 2 provides cell viability assay results plotted as a function of relative cell viability (%) with concentration for HCT116 (cancer cell line), MCF-7 (cancer cell line) and L929 cells (control) after treatment with the conjugate as per an exemplary embodiment of the present disclosure. n=3, error bars: mean+SD; p<0.005.

FIG. 3A-FIG. 3C provide results of in vivo biodistribution and toxicity of the conjugates in C57BL/6J mice models, wherein the conjugates are as per an exemplary embodiment of the present disclosure.

FIG. 3A is an in vivo multispectral image of the conjugate with dye in two different mice models (control and treated) at 810 nm λₑₘ.

FIG. 3B is a graph of a quantitative analysis of percentage hemolysis induced (%) in murine systems by the conjugates at various concentrations ranging from 0 to 100 μg/mL. Error bars, mean+SD; p<0.05.

FIG. 3C depicts results from histological studies of the conjugate in two different mice models (control and treated) post treatment depicted as images from hematoxylin and eosin staining of different organs (the brain, heart, stomach, kidneys, lungs, liver, and spleen). Images were taken at ×20 magnification.

FIG. 3D-FIG. 3G depict quantitative indirect assessment of renal functions estimated through colorimetric tests and recorded as a change in concentration (mg/dl) of Urea (FIG. 3D), Creatinine (FIG. 3E), and hepatic functions Bilirubin (FIG. 3F), and ALP, SGOT, and SGPT (FIG. 3G) in control and treated mice cohorts.

FIG. 4A-FIG. 4F provide results of tumor arrest and regression in the autochthonous BRCA2/p53 knockout breast cancer models administered with conjugates as per an exemplary embodiment of the present disclosure.

FIG. 4A is a schematic diagram of a mammary tumor, constituting a tumor necrotic core (TC) and where the intratumoral injections are given at the tumor periphery (TP).

FIG. 4B depicts mammary tumor growth arrest images in conditional BRCA2/p53 knockout mouse model upon treatment with the conjugate.

FIG. 4C depicts Kaplan-Meier survival plots of the conjugate (EphB4-shRNA+nanoconjugate) treated cohorts; and control cohorts−scrambled shRNA+NPs, shRNA alone, CU NPs alone, and untreated mice (control).

FIG. 4D depicts the tumor retardation curve for the cohorts.

FIG. 4E depicts the immunohistochemistry of Ephb4, β-catenin, and c-Myc in wild type, untreated, and conjugate treated cohort sections (tumor periphery (TP) and tumor core (TC)). Images were developed in ×20 magnification.

FIG. 4F depicts the quantification of the area of degradation through the Image J software for Ephb4. Error bars, mean+SD; ***p<0.001.

FIG. 5A-FIG. 5C depict suppression of intestinal tumorigenesis in AhCre-ErTApcfl/fl murine models administered with conjugates as per an exemplary embodiment of the present disclosure.

FIG. 5A depicts Kaplan-Meier survival curves of mice treated with the conjugate (EphB4-shRNA+nanoconjugate) and different treatment cohorts-scrambled shRNA+NPs, shRNA alone, CU NPs alone and untreated mice (control).

FIG. 5B depicts images from hematoxylin and eosin staining of gut sections from the Apc-deficient mice treated with the conjugate compared with other controls. The images were developed in ×20 magnification.

FIG. 5C depicts quantification of the area of degradation through the Image J software. Error bars, mean+SD.

### DETAILED DESCRIPTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weights, percentages, ratios, and so forth, to describe and claim certain embodiments and are to be understood as being modified in some instances by the term “about.” Accordingly, in some embodiments, the numerical parameters set forth in the written description and attached claims are approximations that can vary depending upon the desired properties sought to be obtained by a particular embodiment. The numerical values presented in some embodiments may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as “comprises” and “comprising,” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the claims presented herein. No language in the specification should be construed as indicating any non-claimed element as being essential to the practice of the invention or any claimed embodiment thereof.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the claims, unless explicitly stated otherwise.

The headings and abstract provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As described herein, the term ‘effective amount’ refers to the amount of the conjugate or composition required to bring about a change or improvement in a subject without side effects or overdosing.

The term, “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young, the term mammal includes animals such as cat, dog, rabbit, bear, fox, wolf, monkey, deer, mouse, pig and human.

The term, ‘management’, or ‘treatment’ as used herein refers to alleviate, slow the progression, attenuation, prophylaxis or as such treat the existing disease or condition. Treatment also includes treating, preventing development of, or alleviating to some extent, one or more of the symptoms of the diseases or condition.

As used herein, the term ‘immobilized’, ‘immobilizing’, ‘complexed’ or ‘complexing’ refers to the physical or chemical attachment of a molecule on at least one point on the surface of another molecule.

Aspects of the present disclosure provide an RNAi-nanoparticle conjugate as an effective drug delivery system for management of cancer, including but not limited to, colon cancer, breast cancer, lung cancer, colorectal cancer, pancreatic cancer, prostate cancer, among others.

In an embodiment, the present disclosure provides an RNAi-nanoparticle conjugate comprising a hydrophobic active ingredient; a polysaccharide; an RNA interference (RNAi) silencer EphB4-shRNA; and a coating agent; wherein the active ingredient is encapsulated by the polysaccharide to give a Nep; the RNAi silencer is immobilized on the Nep and coated by the coating agent.

In an embodiment, the active ingredient may be any well-known chemical, biological or naturally occurring ingredient, including but not limited to, anti-cancer ingredients selected from curcumin, resveratrol, vincristine, luteolin, quercetin, piperine, berberine; monoclonal antibodies including siltuximab, tositumomab, or herceptin; anthracyclines including doxorubicin, daunorubicin, bleomycin, orepirubicin; sorafenib, erlotinib, cisplatin, oxaliplatin, carboplatin, or combinations thereof. In particular embodiments, more than one active ingredient may be present in the conjugate.

In a preferred embodiment, the active ingredient is curcumin. Curcumin, a bioactive spice from Curcuma longa, has been used since a long time to treat various chronic diseases, including cancers. Curcumin possesses potent anti-carcinogenic, anti-inflammatory, and antioxidant properties making it one of the best studied nutraceuticals for cancer therapeutics. However, its poor water solubility and bioavailability prevent its use as an ideal therapeutic molecule.

In an embodiment, the RNA interference (RNAi) silencer EphB4-shRNA is EphB4-shRNA-800. The RNA interference (RNAi) silencer may be incorporated in a vector. In particular embodiments, the vector may be a plasmid.

In an embodiment, the polysaccharide may be selected from, but is not limited to, chitosan, ethyl cellulose, carboxymethyl cellulose, hydroxyl propylmethyl cellulose, methylcellulose, ethyl cellulose, pectin, carrageenan, hyaluronic acid, guar gum, sodium alginate or combinations thereof.

The active ingredient is encapsulated in the polysaccharide followed by complexing of nucleic acids of RNA with the polysaccharide, particularly multiple attachments are preferred. Therefore, in one of the preferred embodiments, chitosan which is a biodegradable, non-toxic, and mucoadhesive cationic polymer is used as the polysaccharide, particularly for the active ingredient curcumin. Moreover, chitosan is found to enhance the effectiveness of absorption of mucosal cells present in the intestine at neutral pH.

In an embodiment, the ratio of the active ingredient to polysaccharide may be about 1:1 to about 1:5, preferably about 1:1 or 1:2. In some embodiments, very high amounts of polysaccharide may decrease entrapment and loading efficiency due to increase in viscosity.

In another embodiment, the ratio of Nep to RNAi silencer EphB4-shRNA is in the range of about 25:0.1 to about 25:1, preferably the range is about 25:0.8 to 25:1.

Furthermore, targeting the conjugates in the colon through the oral route requires bypassing the harsh conditions of the stomach (pH 1.2-2.0). The coating agent protects the conjugate from enzymatic and hydrolytic degradation in the stomach and enhances intestinal uptake of the conjugate. In an embodiment, the coating agent may be any well-known coating agent selected from, but not limited to, polymethacrylate based copolymers including methyl acrylate, methyl methacrylate, methacrylic acid, [2-(dimethylamino) ethyl methacrylate], or 3-hydroxyethylmethacrylate; cellulose derivatives including cellulose acetate, hydroxypropyl methylcellulose, or hydroxypropyl cellulose; or combinations thereof. In a preferred embodiment, the coating agent is a polymethacrylate based copolymer, in particular embodiments, it is Eudragit®. In particularly preferred embodiments, Eudragit® S-100, an anionic pH-sensitive polymer, may be used as a protective coating to prevent the active from degradation in the stomach and the subsequent release of the active in the colon region where the pH is 5.5 to 7.4, low alkaline to neutral.

In an embodiment, the conjugate is nanosized with a size in a range of about 50 to 500 nm. In a preferred embodiment, the conjugates are characterized by having particle size in the range of 110-230 nm. Nanoparticles with their enhanced permeability and retention (EPR) effect overcome the limitations of RNAi-based therapies and demonstrate enhanced cellular uptake. With their specific complex loading capacity, polysaccharides in nanoparticles can target RNAi molecules to tumor tissues by protecting them from enzymatic degradation during transportation.

In an embodiment, the coating agent may be coated with a thickness of about 10 to 50 nm, preferably about 20-30 nm.

In an embodiment, the Neps may be optionally cross-linked with divalent cations and their salts including but not limited to, calcium chloride, or magnesium chloride; TPP (tripolyphospate) or combinations thereof, before immobilization with RNAi. To analyze the progress of the conjugate in the systemic environment, a fluorescent agent may be encapsulated in the conjugate.

The conjugates of the disclosure are biocompatible, non-toxic and exhibit no adverse side effects or systemic toxicity. They specifically deliver the active ingredient and RNAi silencer to the site of tumor. Thus, it is site-specific and gives effective uptake. The RNAi silencer silences Ephb4receptor using RNAi technology to exhibit anti-cancer activity and the active ingredient also provides anti-cancer activity. The RNAi lacks safe delivery system while the NPs lack specific targeting; the conjugate possesses a synergism that gives effective anti-proliferative activity at the targeted site of action.

In a preferred embodiment, the present disclosure provides an RNAi-nanoparticle conjugate comprising curcumin; chitosan; an RNA interference (RNAi) silencer EphB4-shRNA; and a polymethacrylate based copolymer; wherein the curcumin is encapsulated by chitosan to give a Nep; the RNAi silencer is immobilized on the Nep and coated by the polymethacrylate based copolymer.

The conjugate of the present disclosure brings together the synergistic effect of an active ingredient such as curcumin with gene silencing using shRNA in solid tumors. The present disclosure thus combines the effectiveness of the active ingredient and the RNA interference approach for a site-specific and effective therapeutic outcome. It provides an approach for using non-synthetic nanomaterials with RNA interference in biological systems for cancer therapeutics.

In an embodiment, the present disclosure provides an RNAi-nanoparticle conjugate for use in the treatment of cancer.

In another embodiment, the present disclosure provides use of an RNAi-nanoparticle conjugate for management of cancer.

The conjugate is particularly suitable to silence the Ephb4 receptor using the RNAi approach in breast and colon cancers. It provides the combinatorial effect of the active ingredient and the silencer EphB4-shRNA. The nanoarchitecture of the present disclosure, specifically targets the overexpressed oncogenic Ephb4 mRNA, particularly in breast cancer cells, even more particularly in the Brca2/p53−/− mammary cancer cells and Apc−/− of colon cancer cells.

In an embodiment, the present disclosure provides a pharmaceutical composition comprising an RNAi-nanoparticle conjugate comprising a hydrophobic active ingredient; a polysaccharide; an RNA interference (RNAi) silencer EphB4-shRNA; and a coating agent; wherein the active ingredient is encapsulated by the polysaccharide to give a Nep; the RNAi silencer is immobilized on the Nep and coated by the coating agent; and one or more pharmaceutically active excipient(s).

In an embodiment, the excipient may be selected from any of the well-known excipients in the art, including but not limited to, binders, disintegrants, diluents, fillers, lubricants, solubilizers, enteric polymers, sustained release polymers, coloring agents, flavoring agents, sugars, stabilizers, surfactants, gelling agents, or combinations thereof.

In an embodiment, the composition may be formulated suitably into a tablet, elixir, capsule, syrup, powder, granules, solution, suspension, oil, suppositories, paste, or combinations thereof. In an embodiment, the composition may be administered intravenously, intramuscularly, intracranially, dermally, transdermally, orally, rectally, nasally, or combinations thereof.

For the production of oral dosages form of the conjugate such as pills, tablets, coated tablets and hard gelatin capsules, it is possible to use, for example, lactose, corn starch or compounds thereof, gum arabica, magnesia or glucose, etc. Pharmaceutically acceptable excipients that can be used for soft gelatin capsules and suppositories are, for example, fats, waxes, natural or hardened oils, etc. Suitable pharmaceutically acceptable excipients for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the said solvents.

In another embodiment, the pharmaceutical compositions normally contain about 1% to 99%, for example, about 5% to 70%, or from about 10% to about 30% by weight of the conjugate.

In a preferred embodiment, the composition may be administered orally.

In a preferred embodiment, the composition is a solution or suspension administered intravenously at the site of tumor. The composition specifically suppresses the Ephb4 receptor and its downstream genes thereby affecting the Wnt signaling pathway.

In an embodiment, the composition may further comprise one or more other therapeutic agents. This includes co-administration of a conjugate of the present disclosure with the other therapeutic agent or treatment as either a single combination dosage form or as multiple, separate dosage forms, administration of the conjugate of the present disclosure first, followed by the other therapeutic agent or treatment and administration of the other therapeutic agent or treatment first, followed by the conjugate of present disclosure.

In an embodiment of the present disclosure, the other therapeutic agent may be any agent that is known in the art to treat, prevent, or reduce the symptoms of a disease or disorder. The selection of other therapeutic agent(s) is based upon the particular disease or disorder being treated. Such choice is within the knowledge of a treating physician. Furthermore, the additional therapeutic agent may be any agent when administered in combination with the administration of a conjugate of the present disclosure provides benefit to the subject in need thereof. In a particularly preferred embodiment, the other therapeutic agent may be a chemotherapeutic agent.

In another embodiment, the present disclosure provides a method for synthesis of an RNAi-nanoparticle conjugate, the method comprising the steps of: (a) fabricating a Nep comprising a hydrophobic active ingredient encapsulated by a polysaccharide; (b) complexing the Nep with an RNA interference (RNAi) silencer EphB4-shRNA at a temperature in a range of about 40-60° C.; and (c) coating with a coating agent to give the conjugate.

In an embodiment, the conjugate are nanoparticles.

In an embodiment, fabrication of the Nep may be performed by dispersing the active ingredient in the polysaccharide and fabricating into nanoparticles or by fabricating the active ingredient into nanoparticles followed by encapsulation to give the Nep.

In an embodiment, fabrication of NPs may be done by any well-known method in the art. This includes desolvation, microwave irradiation, chemical vapor deposition, sol-gel, co-precipitation, template synthesis, electro spraying, ion sputtering, sonochemical, or combinations thereof.

In an embodiment, the method comprises the steps of: (a) dispersing the active ingredient in the polysaccharide; (b) fabricating the Nep from the dispersion;(c) complexing the Nep with the RNA interference (RNAi) silencer EphB4-shRNA at a temperature in a range of about 40-60° C.; and (d) coating with the coating agent.

In a preferred embodiment, the method comprises the steps of: (a) dispersing curcumin in chitosan; (b) fabricating the Nep from the dispersion by electrospraying;(c) complexing the Nep with the RNA interference (RNAi) silencer EphB4-shRNA at a temperature in a range of about 40-60° C.; and (d) coating with polymethacrylate based copolymer. In particularly preferred embodiments, electro spraying is used for the fabrication of Nep with high yields and reproducibility.

In another embodiment, curcumin may be dispersed in chitosan after formation of curcumin NPs. Accordingly, in one particular embodiment, the curcumin is downsized to nanocurcumin (curcumin nanoparticles) by sonication. The curcumin NPs, being negatively charged, interact with positively charged polymers such as chitosan. The desolvation approach is usually used for the encapsulation of curcumin with polysaccharides. However, following this method for curcumin, led to low yield of the NPs compared to electrospraying.

The RNAi silencer EphB4-shRNA may be complexed with the Neps by any method known in the art, preferably by heating and mixing the heated solutions. Similarly, the coating may be performed by simply mixing the coating agent with the RNAi silencer complexed Nep.

In an embodiment, the present disclosure provides a method of silencing Ephb4 receptor in a subject by administering an effective amount of the RNAi-nanoparticle conjugate or the pharmaceutical composition as recited above.

In an embodiment, the present disclosure provides a method of management or treatment of cancer in a subject by administering an effective amount of the RNAi-nanoparticle conjugate or the pharmaceutical composition as recited above.

The effective amount will depend upon a variety of factors including the activity of the conjugate, the route of administration, the time of administration, the rate of excretion of the particular conjugate being administered, the duration of the treatment, other concurrently administered drugs, compounds and/or materials, the age, sex, weight, condition, general health and prior medical history of the subject being treated, and like factors well known in the medical arts.

In an embodiment, the cancer may be selected from colon cancer, breast cancer, lung cancer, colorectal cancer, pancreatic cancer, prostate cancer, testicular cancer, bone cancer, or combinations thereof; particularly the cancer is breast cancer or colon cancer. In particular embodiments, the subject is human.

The conjugate or composition inhibits Ephb4 receptor or silences the gene thereby enabling arrest of tumor growth and the subsequent regression of tumor volume over a period of time.

In particular embodiments, the conjugate or composition helps in regression of tumor size and increasing survival rates in a subject.

In an embodiment, the conjugate or composition suppresses and reverses the Wnt induced deregulated state or reverse intestinal tumorigenesis. In some embodiments, the conjugate or composition may result in a concomitant decrease in the expression levels of the Ephb4 and c-Myc and re-localization of β-catenin from the nucleus to the cytoplasm in breast and colon cancer. In some embodiments, the conjugate or composition increases Nf-κB expression that increases survival response.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

Embodiments of this disclosure are further explained in the form of following examples. However, it is to be understood that the following examples are merely illustrative and are not to be taken as limitations upon the scope of the invention or of the appended claims.

### Materials

Ephb4-shRNA was obtained from Origene, USA. Chitosan (190-310 kDa) was procured from Sigma-Aldrich, USA. Eudragit S-100 was a gift sample from Evonik Industries, Germany. Crystalline curcumin was procured from S. D. Fine, India. Calcium chloride and cyanine 7.5 NHS ester dye were procured from Spectrochem, India and Lumiprobe, USA, respectively. FBS (fetal bovine serum), DMEM (Dulbecco's modified Eagle's medium), MTT (3-(4,5-dimethylthiazol-2-yl)-2,5-diphenyltetrazolium bromide) were purchased from Invitrogen, USA. L929, HCT116, and MCF-7 cell lines were obtained from the cell repository of the National Centre for Cell Science (NCCS), India. Primary and secondary antibodies were purchased from Abcam, UK. Immunohistochemistry kits were procured from the DAKO Envision system, USA. The solvents and reagents of analytical grade were obtained from a local vendor. The liver function test (LFT) and renal function test (RFT) kits were purchased from Coral Clinical Systems, India. The zeta potential and particle size distribution were analyzed using a 90 Plus Brookhaven Instruments Corp, PALS zeta potential analyzer, USA. The morphological characterization was done using ESEM Quanta 200 3D, FEI, USA and FTIR PerkinElmer spectrometer I, FT-IR diffused reflectance (DRIFT) mode, USA. The thermal behaviors were recorded using a differential scanning calorimetry (DSC) instrument (Model Q10 DSC, TA instrument, USA).

### Example 1

### Preparation of a Fluorescent Nanoencapsulate

Curcumin (CU)-cyanine-7.5 NHS dye dispersion was prepared according to the reported article (RSC, Nanoscale, A. Kumar, A. Singam et al). Furthermore, the respective 1 and 2 wt % of chitosan (CS) solutions of pH 5.0 were prepared in 2% (v/v) acetic acid solution by stirring for 6 h. Equal volumes of CS and CU-cyanine-7.5 NHS dye dispersion were added slowly drop-wise under high-speed stirring to make a CU-CS suspension. The CU NP concentration was fixed at 1 w/v %, and the CS concentration was varied (from 1 w/v % to 2 w/v %) to attain various formulations. To crosslink, the individual formulations were electrosprayed vertically in 100 mL of calcium chloride (5 wt %) solution using an electrospinning unit (ESPIN Nano, PECO, India). The parameters for electrospraying were optimized at a voltage of 24 kV with a flow rate of 0.5 mL h⁻¹. The distance between the tip of the needle and the collector was maintained at a distance of 15 cm. The electrosprayed dispersion was centrifuged at 16,000 rpm for 24 min at 12° C. to pelletize the Neps. The obtained pellet was dispersed in deionized water (DI) and washed twice, followed by lyophilization and characterizion.

### Example 2

### Immobilization of Ephb4-shRNA on Nanoencapsulate

Complexes of Ephb4-shRNA with the respective ratios of Neps were formed with two different weight ratios, 25:0.8 and 25:1.0 [(CU-CS) and shRNA]. The respective Neps with different ratios of Ephb4-shRNA were heated separately for 12 min at 55° C. and then the solutions were mixed and vortexed for 30 seconds to obtain the respective CU-CS-shRNA NPs.

### Example 3

### Preparation of Conjugates

The NPs of Example 2 were coated with Eudragit S-100(ES) using 1 wt % ES solution of pH 5, which was prepared using a solvent mixture of ethanol and acetone in a 2:1 ratio. From this solution, 200 μL was added dropwise to the dispersion of 800 μL of the CU-CS-shRNA NPs under stirring to form the conjugates.

### Example 4

### Characterization

### Shape and Zeta Potential

The morphology of the conjugates and CU-CS NPs was examined by ESEM and transmission electron microscope (TEM). The lyophilized NPs were dispersed and diluted (10×) in Millipore water to form a suspension that was later cast on a silicon wafer and sputter-coated with gold for ESEM analysis. Similarly, NP dispersions were prepared, drop cast on a copper grid, and stained with 1 wt % uranyl acetate dye for TEM analysis (FEI Tecnai TF20 200 kV FEG high-resolution Transmission Electron Microscope, USA).

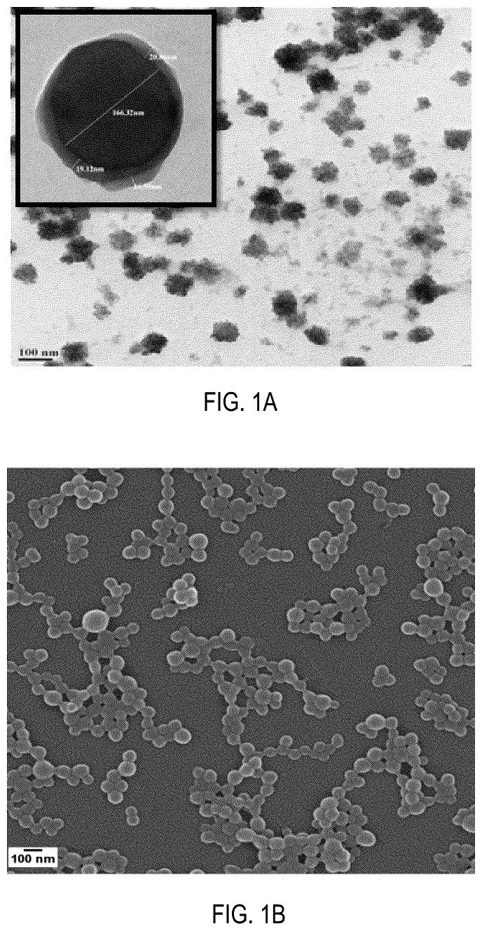

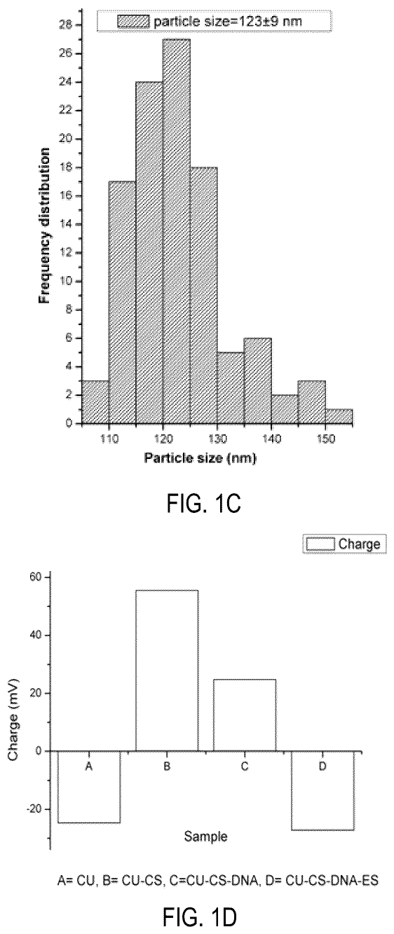

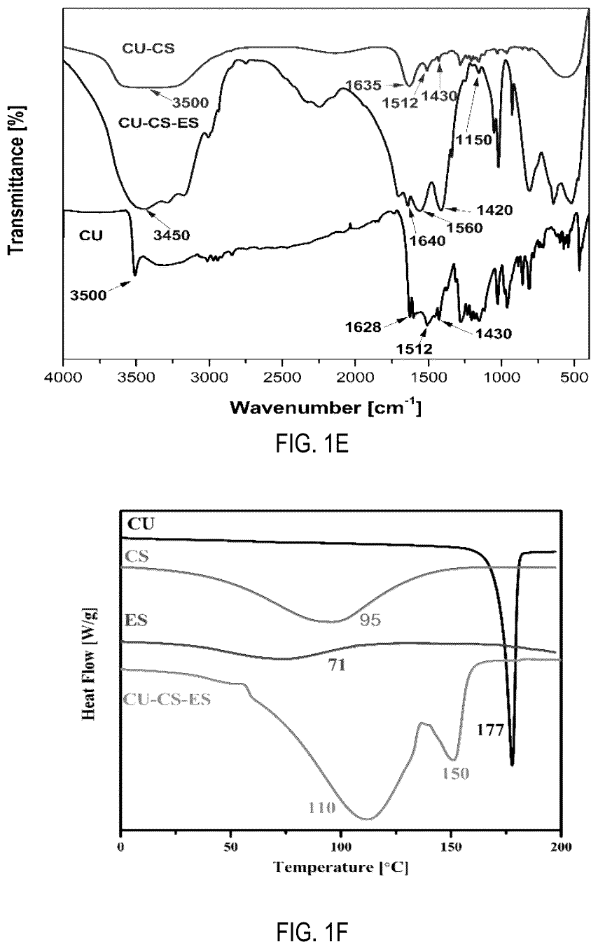

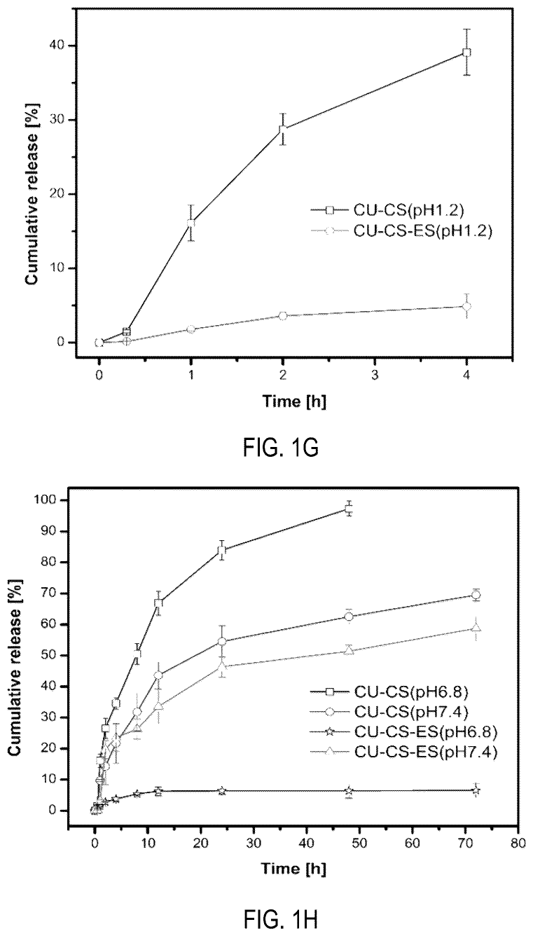

The size, shape, and distribution of the formulated CU-CS NPs and conjugates were in the range of 100-200 and 110-230 nm diameter, respectively, with a distorted spherical to square morphology as shown in FIG. 1A-FIG. 1C. Coatings of chitosan on CU NPs were observed in TEM images, where coating with ES changed the surface morphology of CU with a thickness of the polymer coating between 20-30 nm. Nanoencapsulation of negatively charged CU was carried out using the individual polymer, CS, followed by coating with ES. The charge of the CU particles before and after coating with polymers was confirmed by zeta (ζ) potential measurements. CU possessed a net negative charge of −24.78 mV, which upon interaction with a cationic polymer, CS (+29.1 mV), yielded positively charged particles of +51.52 mV. The increase in the positive charge was attributed due to the crosslinking of NPs with Ca++ ions. With an increase in the ratio of CS, the positive charge of the NPs increased in a concentration-dependent manner.

With the progress of Ephb4-shRNA plasmid conjugation, the net positive charge on NPs decreased to 24.83 mV. This complex formation of CU-CS-shRNA NPs was aided by the positively charged amine groups of CS. Furthermore, the complexed NPs recorded a decrease in net positive charge when coated with anionic ES to give the conjugates (FIG. 1D).

### Entrapment and Loading Efficiency

The entrapment and loading efficiency of the Neps were calculated according to reported literature. Briefly, 2 mg of Neps (1:1 ratio of CU:CS) was dispersed in 2 mL of methanol by sonication for 2 min. The resulting dispersion was centrifuged at 16,500 rpm for 35 min at 12° C. and collected from the supernatant. The unentrapped CU concentration present in the supernatant was calculated using a standard calibration curve of CU at λₘₐₓ of 423 nm, which was analyzed using a UV-Vis spectrophotometer (UV 1601PC UV spectrophotometer, Shimadzu, Japan). The percent of entrapment efficiency (EE) and loading efficiency (LE) were estimated using Equations reported in literature (RSC, Nanoscale).

The EE for different Neps of CU-CS (1:1 and 1:2) was 76.67 and 68.21%, respectively. The loading efficiency was 38.3 and 22.85% for the respective Neps. It was observed that with an increase in CS concentration, the entrapment and loading efficiency of CU were reduced, which could be attributed to the hindrance of CU entrapment as a result of an increase in the viscosity of CS solution.

### Gel Retardation Assay

Gel retardation assay was used to validate the immobilization of the Ephb4-shRNA plasmid to the CU-CS Neps. Two different ratios of CU-CS NPs to Ephb4-shRNA plasmid were prepared (25:0.8 and 25:1.0) and were tested for their electrophoretic mobility in 1% of agarose gel. It was observed that the control lane containing free nucleic acids migrated down the gel without hindrance, while the lane with the Ephb4-shRNA complexes did not show the migration. Additionally, the 25:0.8 ratio exhibited better conjugation efficiency than the 25:1.0 ratio.

While different ratios of CU-CS Neps were formulated and checked for their charge and shRNA plasmid retention, based on the suitability, the 1:2 ratio of CU-CS Nep was selected for pre-clinical evaluation.

### FTIR

The FTIR spectra recorded were in the range of 400 to 4000 cm⁻¹ with an average of 10 scans per sample. The conjugate consisted of the respective components CU, CS, and ES possessing the characteristic functional groups and their sequential additions were confirmed by FTIR. The individual peaks of the respective components were first observed to understand the functional changes before and after the fabrication of conjugates. The IR spectrum of the conjugate reported the changed peak at 1640 cm⁻¹, which could be attributed to the formation of a carboxylate bond between the —NH⁺ groups of CS and the COO⁻ groups of ES which are as per earlier literature. These findings confirmed the encapsulation of CU-CS NPs with ES in the conjugate (FIG. 1E).

### Differential Scanning Calorimetry (DSC)

Respective samples of ˜5 mg were used for recording the thermograms. Each sample was crimped in an aluminum pan and placed in the sample chamber of a DSC that was equilibrated to −80° C. for 2 min and exposed to the heating and cooling cycles. In the first cycle, the sample was heated to 200° C. at a rate of 10° C. min⁻¹. In the second cycle, the sample was quenched to −80° C. at a rate of 100° C. min⁻¹. In the third cycle, the sample was heated from 0 to 200° C. at a rate of 10° C. min⁻¹. The DSC studies were done under a dry nitrogen atmosphere at a purging rate of 50 mL min⁻¹.

To understand the coating strength of ES on the CU-CS NP system, thermal analysis by DSC was performed for CU, CS and ES, and conjugates as shown in FIG. 1F. For CU-CS-ES conjugates, two broad endothermic peaks were observed at 110° C. and 150° C. The Tm of CU decreased because of the interactions between the CS and CU. The cross-linking of CU-CS with CaCl₂ resulted in a shift of the endothermic peak (T_{g}) from 95° C. to 110° C. which could be attributed to an increase in the rigidity of the conjugate. As reported in the earlier studies, the shift in the peak of CU to a lower temperature indicated the disordered crystalline phase in the final conjugates. Thus, the shifting and broadening of the CU peak supported and validated the encapsulation of CU by CS and ES polymers in the conjugate.

### Example 5

### In Vitro Curcumin Release Studies

The CU release studies for the developed conjugates were performed by the direct dispersion method in different buffers, HCl-KCl (hydrochloric acid-potassium chloride 0.1 M, pH 1.2), PBS (phosphate buffered solution, 0.1 M, pH 6.8), and PBS (phosphate buffered solution, 0.1 M. pH 7.4) to mimic the different conditions that exist in the alimentary/gastrointestinal canal. The physiological pH of the stomach is 1.2 (acidic) whereas the pH of the gut ranges from 6.8 to 7.5.The release of CU from CU-CS Neps and CU-CS-shRNA-ES NPs or conjugates was determined according to reported protocol. 5 mg of the respective lyophilized NPs were dispersed separately in 20 mL of different buffers as mentioned above. A duplicate set of 1 mL each was aliquoted from the above respective dispersions. Release kinetics was monitored at 37° C. in a water bath shaker (50 rpm) for a period of 4 h in the buffer of pH 1.2. Similarly, the release studies were done in the respective buffers of pH 6.8 and 7.4 for up to 72 h. After the designated time intervals, the sample tubes were taken out and centrifuged at 1200 rpm for 3 min to collect the pellet of released CU. Later, the collected CU pellet was dissolved in 1 mL of methanol and the amount of CU was quantified by a UV-Vis spectrophotometer (UV 1601PC UV spectrophotometer, Shimadzu, Japan) at a wavelength of 423 nm.

Different pH values chosen helped in understanding the ability of the conjugate to reach the targeted site without degradation. It was observed that at 1.2 pH after 4 h of release studies, the amount of CU released from the Nep system without ES coating was 39%, which is considered as burst release, whereas the conjugate recorded only ˜5% CU release (FIG. 1G). This decrease in the quantity of CU release is expected to be due to the ES coating as a result of the protonation of the COO⁻ groups of ES which enabled the protection of chitosan from degradation at the acidic pH of 1.2. CU release at pH 7.4 for NPs of CU-CS and CU-CS-ES conjugates after 8 h of release, was 32% and 26%, respectively (FIG. 1H), which indicated a sustained release mechanism.

Furthermore, a continuation of CU release for 72 h at the same pH recorded 70 and 59% from CU-CS Nep and CU-CS-ES conjugates, respectively. At this pH, the carboxyl groups of ES were ionized leading to the dissolution of ES thereby exposing CS coated CU NPs. At 6.8 pH (FIG. 1H), the entire CU was released from CU-CS Nep alone within 48 h, whereas the release was only 7% from CU-CS-ES conjugate, even after 72 h of release. The diffusion of CU and swelling of chitosan at pH 6.8 resulted in high CU release from CU-CS Nep, while the stability of ES at pH 6.8 did not lead to any CU release from CU-CS-ES conjugates.

### Example 6

### MTT Assay

The cytotoxicity assay, MTT for CU-CS-Ephb4-shRNA-ES conjugate was done in normal L929, human colorectal carcinoma HCT116 and breast adenocarcinoma MCF-7 cell lines for 48 h. The protocol followed for the MTT assay is reported in published manuscript (RSC, Nanoscale).

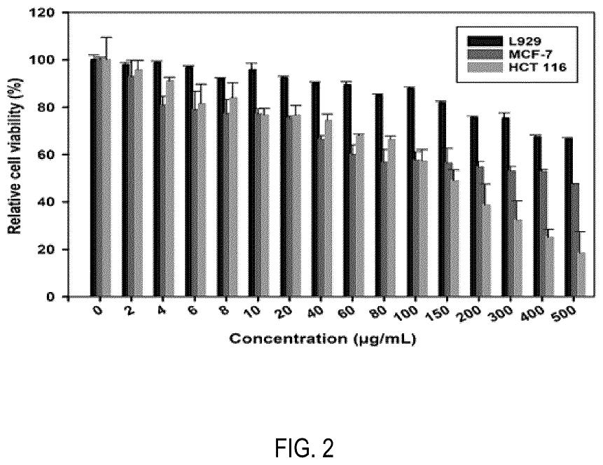

The percentage relative cell viability of L929, HCT116 and MCF-7 cells after incubation with varying concentrations of conjugates is shown in FIG. 2. The conjugates were observed to inhibit the proliferation of both cancer cell lines in a concentration-dependent manner as compared with the controls. The highest toxicity in cancer cell lines (HCT116 and MCF-7) was observed, where the cell viability was reduced by more than 50%. Furthermore, there was no toxicity observed in normal L929 cell lines, demonstrating its specificity against only the cancer cell lines. Thus, the results clearly indicate that the developed CU-CS-Ephb4 shRNA-ES conjugate is potent in reducing colon and breast cancer cell proliferation in vitro.

### Example 7

### In Vivo Studies

The evaluation of the biodistribution and toxicity profiles of the present conjugates in vivo was conducted before validating the therapeutic efficacy. All experiments were performed at the Centre for Cellular and Molecular Biology (CCMB), Hyderabad in compliance with the relevant guidelines of ‘The Committee for the Purpose of Control and Supervision of Experiments on Animals’ (CPCSEA), Government of India. All animal studies were conducted by following the strict ethical guidelines stipulated by institutional guidelines and as per the approval of the Institutional Animal Ethical Committee (IAEC) (project no. IAEC 03/2019).

### Biodistribution

The ES coating around the conjugates protects them from the harsh conditions of the stomach and delivers them effectively in the colon. To analyze the biodistribution of orally delivered conjugates in mice, in vivo multispectral imaging was performed. Wild type mice (C57BL/6J) (n=3) were treated for 15 days by oral gavaging with conjugates with the cyanine 7.5 NHS ester dye.

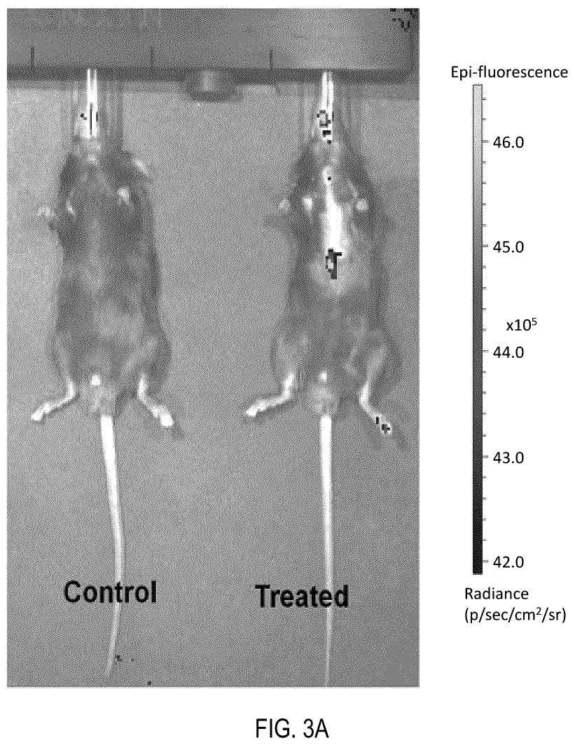

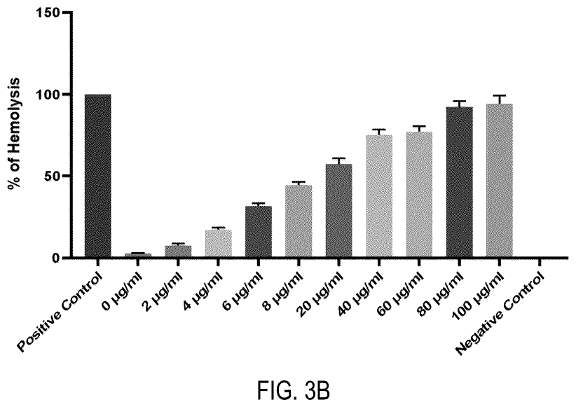

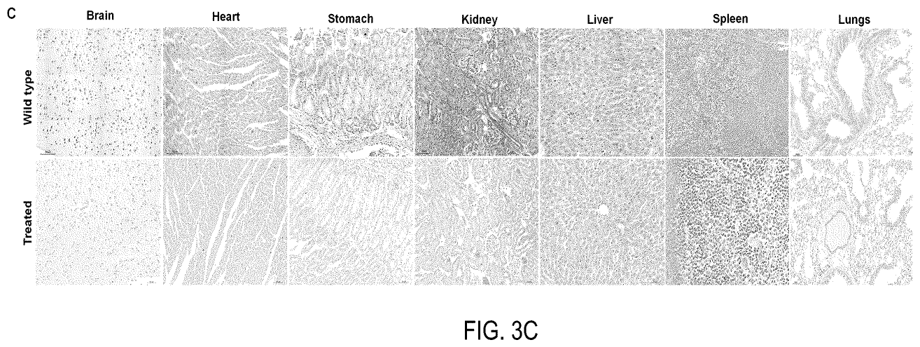

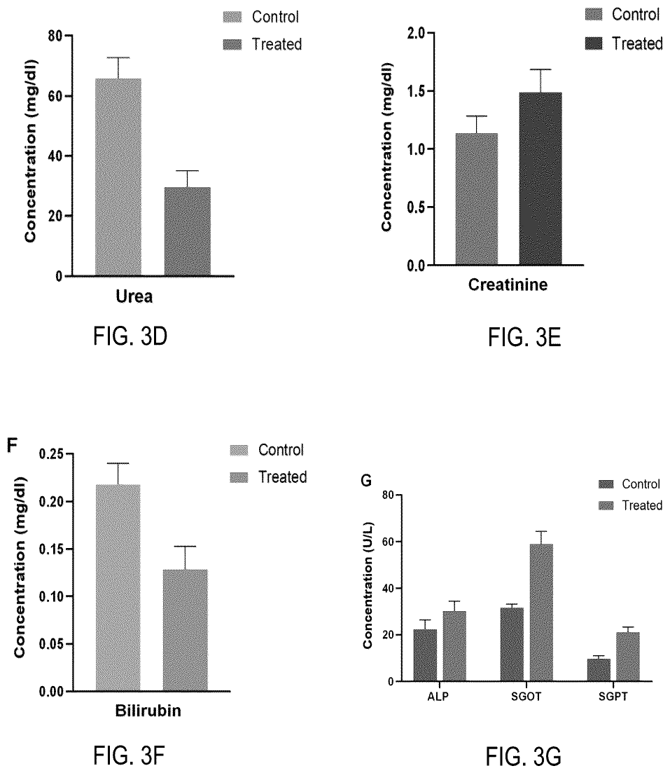

Live imaging tracked the presence of the conjugates at 810 nm λₑₘ, where the first image was taken after the mice were humanely anesthetized using isoflurane after 6 hours of treatment and visualized under a multispectral imager (FIG. 3A).The conjugate was localized basically in the gut region after 24 h of oral administration as compared with the control. Moreover, ex vivo multispectral imaging revealed that the distribution of conjugates after 24 hrs was localized all along the intestinal tract, with higher doses in the regions of small intestines, colon, liver and kidney. Importantly, the presence of the conjugates was not observed in the gall bladder, spleen, heart, lungs and pancreas.

Toxicity profile

The in vivo toxicity of the conjugates was investigated in C57BL/6J healthy mice for a period of 14 days in a sub-chronic study. Accordingly, mice were administered with the conjugate orally at a dose of 1 mg ml⁻¹ body weight, and their behavior (i.e., exploratory activity, eating, sleeping, and neurological symptoms) was carefully monitored. The safety of this drug was tested in the circulatory system using hemolytic assay in murine systems. To examine the hemolytic capacity of the developed conjugates, the hemolytic assay was performed on fresh rodent red blood cells (RBCs).

A 2% v/v suspension of RBCs was prepared. Then dispersions of the conjugates of concentrations 0, 2, 4, 6, 8, 20, 40, 60, 80, 100, 150, 200, 300, 400, and 500 μg mL⁻¹ were incubated with the suspension of RBCs at 37° C. for 1 h (FIG. 3B). The positive and negative controls were Milli-Q water (100% hemolysis) and normal saline (0% hemolysis), respectively. After 1 h of incubation at room temperature, conjugate treated RBCs were spun at 4000 rpm at 4° C. for 10 min and the supernatant was separated. The absorbance was measured at 540 nm using a Microplate Reader (Thermo Scientific, USA).

A concentration of 100 μg ml⁻¹ induced maximum hemolysis among all the tested concentrations of conjugates. Similarly, histological examinations using hematoxylin and eosin staining revealed that major organs like the brain, heart, lungs, stomach, liver, spleen, and kidneys were not exposed to toxicity and there was no inflammation after oral administration of the conjugate when compared with controls (FIG. 3C).

### Liver Function Test (LFT) and Renal Function Test (RFT)

The mice subjects (C57BL/6J) were divided into two different cohorts, namely control and treatment for the liver function test (LFT) and renal function test (RFT). The control group was treated with a buffer and the treatment cohort received 1 mg ml⁻¹ concentration of the conjugate for 2 weeks in a subchronic study.

After treatment, the mice were sacrificed as per ethical guidelines and the blood was harvested in vacutainers. The whole blood was allowed to clot for about 15-30 minutes followed by centrifugation at 1500 g for 15 minutes at 4° C. in a refrigerated centrifuge and the serum was isolated for the assessment. For LFT, the determinations of albumin, total bilirubin, alkaline phosphatase, SGOT, and SGPT were performed and for RFT, urea and creatinine were measured. Each particular test was performed using the mentioned concentrations and ratios as per the manufacturer's instructions. The time, temperature of incubation, and the filter wavelength were accurately followed. The results were analyzed, and the corresponding values were plotted as graphs using GraphPad Prism 9.0.

Secondary assessment of the liver and kidney functions by means of indirect colorimetric tests like LFT and RFT assays exhibited no noticeable changes in all the parameters for the conjugates in the treated cohorts when compared with the controls. The concentrations of urea, creatinine, alkaline phosphatase, ALT, SGPT, and SGOT were well within the reference levels in both groups (FIG. 3D-FIG. 3G). The toxicological results obtained after the oral administration of the conjugates to healthy mice were satisfactory and not detrimental. These results suggested that the conjugates of the present disclosure are not only safe but also bio-compatible in the murine system and thus can be extended to humans after pre-clinical trials.

### In Vivo Efficacy

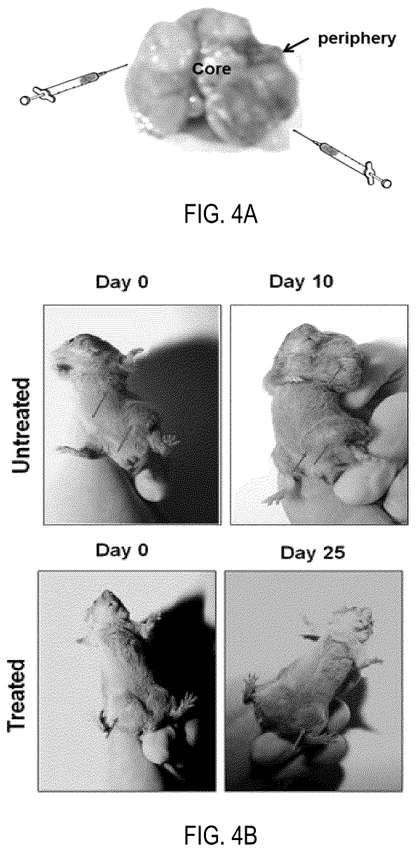

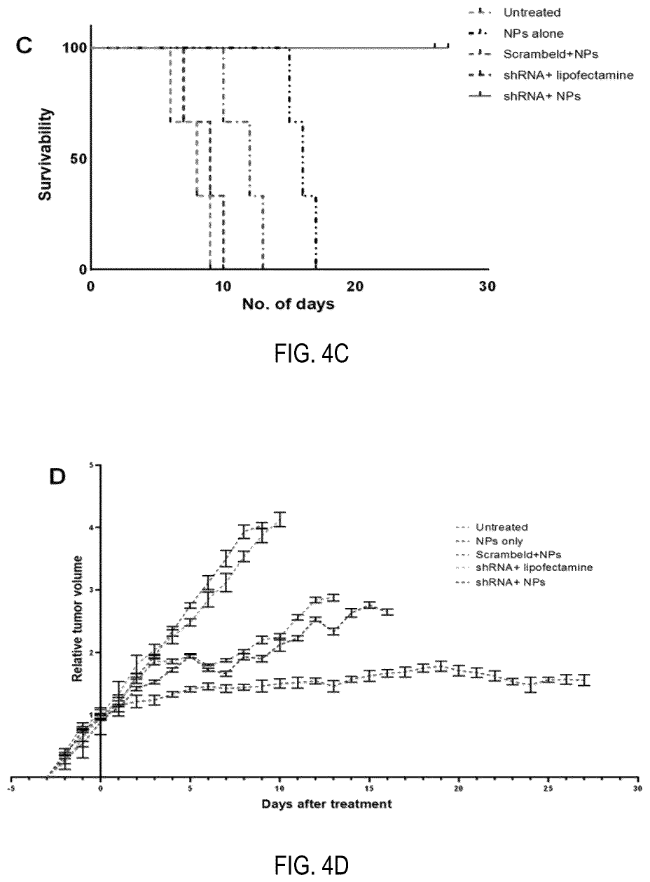

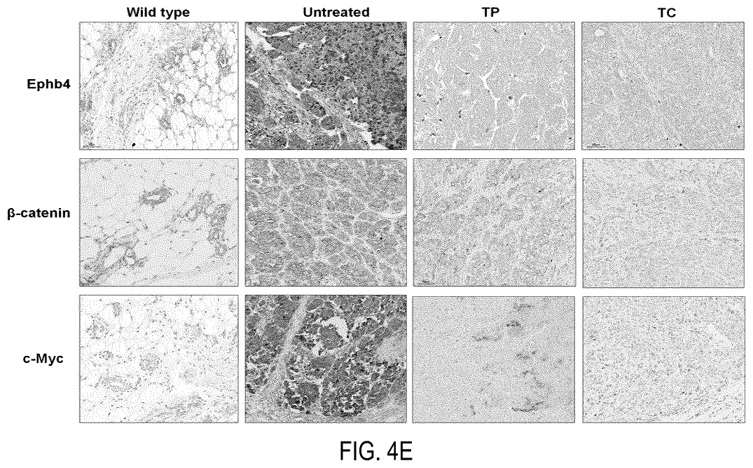

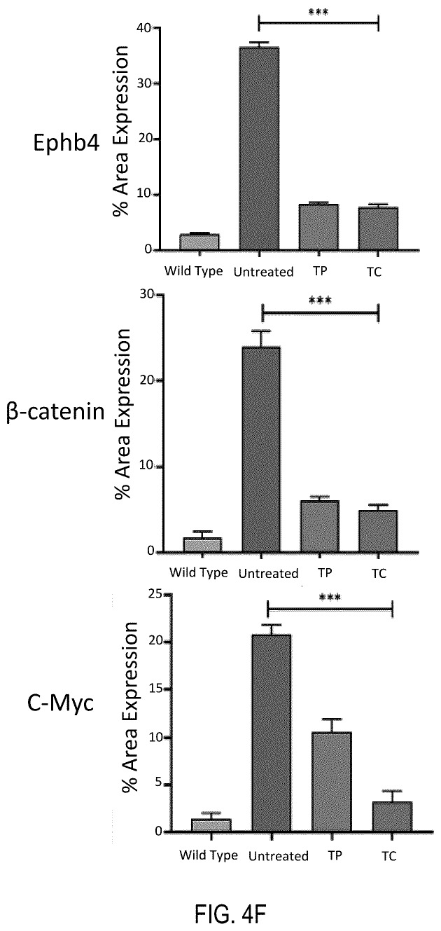

Solid autochthonous tumors are relatively distinct from xenograft models for most treatment regimens and often pose challenges due to their inherent nature of origin and the associated complications with delivery to necrotic tumor cores (FIG. 4A). Therefore, the ability of the conjugates to suppress tumors was investigated in vivo in an established transgenic Blg-cre Brca2/p53 double knockout murine breast cancer model where the mice develop mammary tumors naturally between 6 and 15 months of age. It is well documented in humans that the inheritance of mutations in the susceptibility genes such as BRCA1 and BRCA2 increases the risk of developing breast cancer. The somatic loss of BRCA2 and p53 in mice models mimics human cancers and induces mammary tumors naturally between 6 months to one year of age in any/all of the five mammary glands making it one of the best models akin to human breast cancer. Since it is an autochthonous tumor, regression is a difficult process compared to xenograft models.

To assess the efficacy of the conjugate in solid tumors, intratumoral injections of the conjugates along with appropriate controls were administered directly to tumor periphery. Two different animal knockout models were used in the study to assess the conjugate's efficacy against solid tumors. For evaluating the therapeutic potential of the conjugate in breast cancer murine models, conditional Brca2/p53 knockout under control of Blg-cre transgene was used. Mice developing 0.5 m3 mammary tumors were selected for different cohorts which included conjugates, scr control (scrambled shRNA+NPs), shRNA+lipofectamine, CUNPs only, and untreated (only induction and no treatment). Intratumoral injections of 10 μg of Ephb4-shRNA (in pGFP-V-RS vector) complexed with 250 μg of Nep (CU-CS; in a volume of 50 μL) were given to the treated cohorts along with the respective controls. The mice were given the drugs equally and the tumor sizes were measured every day and selected for survival and other data analyses.

The inducible Apc knockout colon murine models (AhCre-ErTApcfl/fl) develop the crypt progenitor phenotype in the intestine upon induction with β-naphthoflavone, and tamoxifen, demonstrating a progressive knockout, causing mortality of the animal within 8-10 days. To assess the efficacy of the conjugate against colorectal cancer, the conjugates were orally administered to Apc knockout models. The mice subjects were divided into five different cohorts of five mice each and were given the following treatments. The cohorts included those treated with complete conjugates alone with controls:scr control (scrambled shRNA+NPs), shRNA+lipofectamine, CU NPs (NPs only) and untreated (only induction and no treatment).

Six to eight-week-old mice were selected for the study and were given intraperitoneal injections comprising 80 mg kg⁻¹ of β-naphthoflavone and tamoxifen (dissolved together in corn oil 10 mg ml⁻¹ each) once daily for 5 days to induce the recombination of the targeted alleles (knockout). The Apc knockout induced mice were treated with a daily oral dose of 50 μg of Ephb4-shRNA (in pGFP-V-RS vector)-encoding plasmid DNA complexed to 1.25 mg of Nep (in a total volume of 250 μL) from day 1 of induction until the end time point at which the mice survived.

After treatment, the animals were sacrificed as per the ethical guidelines after 180 days by cardiac perfusion (using 4% paraformaldehyde) and tissue collection was performed. The tissues harvested were preserved accordingly for all downstream experiments. The intestines were specifically cleaned in running tap water and 1×PBS, opened up, and fixed in methacam for 48 h. The intestines were then made into a gut roll and preserved in 10% formalin and after the processing were sectioned with 4 μm thickness.

Tumors treated with the complete conjugate showed a marked arrest and subsequent decrease of tumor size compared with different controls (FIG. 4B). Untreated mice show aggressive tumor growth in 10 days but treated mice depict tumor arrest even after 25 days. Moreover, conjugate treated cohorts exhibited an increased median survival response of 35-40 days when compared with the various controls where the mice survived only for 10-15 days. From the Kaplan-Meier survival plots, it can be seen that CU nanoparticles demonstrated median survival of 15-20 days, which was higher than the other controls, as CU has been well established for its potent anti-inflammatory and anti-cancerous attributes (FIG. 4C and FIG. 4D). A prolonged survival of 27 days in conjugate treated cohorts compared to other treated cohorts was noted along with tumor growth arrest.

Immunohistochemical analysis of core tumor sections revealed a significant reduction in the expression levels of the Ephb4 protein in the treated cohorts that resulted in the suppression of tumor growth and therefore increased survival rates. The reduction of Ephb4 levels in the tumor core proved beyond doubt that the administered conjugates from the tumor periphery have penetrated to the core to bring about the suppression and subsequent repression of tumor size.

Onsite delivery of the Ephb4 shRNA plasmid straight into the mammary tumor periphery enabled targeting of the necrotic tumor core, aiding in the effective knockdown of Ephb4, thus enabling the arrest of tumor growth and the subsequent regression of tumor volume over a period of time. This simultaneous suppression and regression of tumor burden within a short time span indicated the dual action of the conjugate involving the anti-inflammatory attributes of curcumin along with the gene silencing of Ephb4 gene by RNA interference (FIG. 4E and FIG. 4F).

The conjugate demonstrated effective reversal of intestinal tumorigenesis in Apc knockout murine models. The Wnt pathway is involved in orchestrating the development and maintenance of the niche in the intestines. Adenomatous polyposis coli (Apc), a tumor suppressor gene in the Wnt pathway, is the main component of the destruction complex that phosphorylates the oncogene β-catenin for degradation. Mutations of the adenomatous polyposis coli gene predispose individuals to familial adenomatous polyposis (FAP) syndrome. Similarly, Apc knockout colon cancer models (Cre+Apcfl/fl) develop altered crypt/villus architecture that mimics human colon cancer and die between one week to 10 days, thus making this robust model ideal for drug testing compared to other existing models. Moreover, in the Apc inducible knockout model, the deletion of the Apc gene results in the unphosphorylated β-catenin getting translocated from the cytoplasm to nucleus. This results in the activation of downstream genes like c-Myc, CD44, etc. that consequently affects the development of an aggressive c-Myc dependent crypt progenitor phenotype across the intestine. This results in the formation of early tumor lesions leading to the death of the animals within 7 to 10 days.

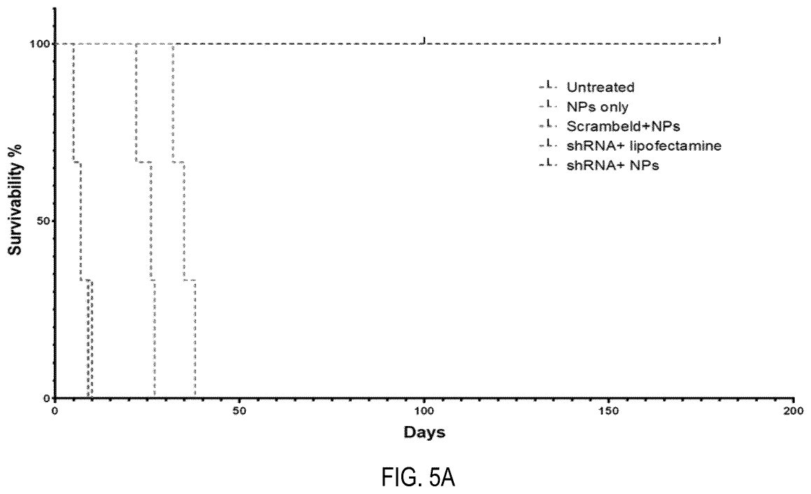

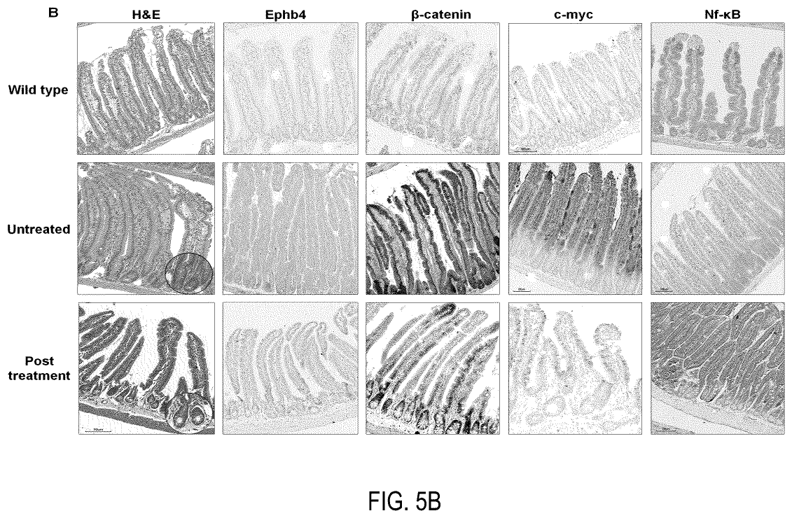

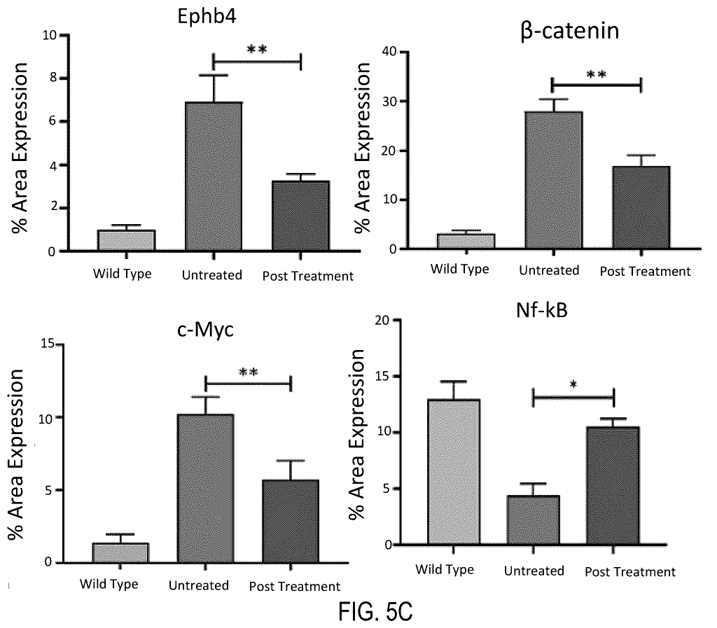

Accordingly, to assess the therapeutic potential of the conjugate of the present disclosure in effective reversal of intestinal tumorigenesis, the bio conjugate was orally administered to target the gut in Apc knockout murine models in an attempt to suppress and reverse the Wnt induced deregulated state. Although the CU NPs exhibited median survival between 40-60 days, the combinatorial approach using RNA interference and CU NPs had a high median survival of 180 days when compared with the controls having median survival in the range of 10-15 days demonstrating surprising synergism (FIG. 5A). Morphological evaluation by hematoxylin and cosin (H&E) staining revealed a return of the crypt structures that were nearly akin to wild type in the treatment cohorts when compared with the untreated sections where the villi appeared to be fused with the crypts exhibiting a crypt progenitor type state. Immunohistochemical results show the site-specific knockdown of target genes Ephb4, β-catenin, c-Myc, and Nf-κb in the treated cohorts as compared to the respective controls.

Immunohistochemical analysis throughout the treatment reflected a reduced expression of Ephb4 levels indicating a successful shutdown of the Ephb4 by the biodrug. The altered crypt progenitor type was no longer identifiable thus bringing about the normal morphology of the crypt-villus axis indicating a reversal of intestinal tumorigenesis. Moreover, β-catenin was re-localized from the nucleus to the cytoplasm during the treatment, indicating a return to a non-Wnt-deregulated state. Increasing experimental evidence has well established the oncogenic role of c-Myc in colorectal carcinoma. Here, treatment with conjugates resulted not only in decreasing the level of Ephb4 proteins but also decreased the expression levels of oncoprotein c-Myc in the test groups compared to untreated controls. Thus, the Ephb receptor tyrosine kinase controls Wnt signaling through its target genes that are active in cell compartmentalization along the crypt axis. Hence, the shutdown of Ephb4 might have resulted in reduced expression of Wnt target genes like β-catenin and c-Myc stimulating the intestine cell niche to return to a near normal state. Nf-κB, a known transcription factor, is responsible for regulating various immune responses and acute inflammation in normal physiological processes. The treatment resulted in increased Nf-κB expression in the treated cohorts which mimicked the wild type state potentiating a strong survival response (FIG. 5B and FIG. 5C).

This prolonged survival due to tumor regression is an indication of the synergistic action of the shutdown of upregulated Wnt genes by RNAi as well as the tumor suppressive action of the curcumin nanoparticles. These results substantiate the importance of recognizing shRNA molecules as more stable compared to siRNA to bring about efficient and consistent gene silencing and also reveal the role of nanotechnology not only as a carrier molecule but also as a therapeutic molecule. These findings supported the high pharmacological potential and synergetic effect of this combinatorial therapy in suppressing and abrogating the intestinal tumorigenesis and helped in returning it to a near wild type state.

Thus, the present disclosure demonstrated that the conjugate efficiently regresses the tumors by effective knockdown of target genes with a concomitant increase in survivability.

what is claimed is:

1. An RNAi-nanoparticle conjugate, wherein the RNAi-nanoparticle conjugate comprises:
a hydrophobic active ingredient;
a polysaccharide;
an RNA interference (RNAi) silencer; and
a coating agent,
wherein the hydrophobic active ingredient is encapsulated by the polysaccharide to form a nanoencapsulate, and the RNAi silencer is immobilized on the Nep and coated by the coating agent.

2. The RNAi-nanoparticle conjugate of claim 1, wherein the active ingredient is selected from curcumin, resveratrol, vincristine, luteolin, quercetin, piperine, berberine, siltuximab, tositumomab, herceptin, doxorubicin, daunorubicin, bleomycin, orepirubicin, sorafenib, erlotinib, cisplatin, oxaliplatin, carboplatin, or combinations thereof.

3. The RNAi-nanoparticle conjugate of claim 1, wherein the polysaccharide is selected from chitosan, ethyl cellulose, carboxymethyl cellulose, hydroxyl propylmethyl cellulose, methylcellulose, ethyl cellulose, pectin, carrageenan, hyaluronic acid, guar gum, sodium alginate, or combinations thereof.

4. The RNAi-nanoparticle conjugate of claim 1, wherein the RNAi silencer comprises EphB4-shRNA-800.

5. The RNAi-nanoparticle conjugate of claim 1, wherein the coating agent is selected from methyl acrylate, methyl methacrylate, methacrylic acid, [2-(dimethylamino) ethyl methacrylate], or 3-hydroxyethylmethacrylate; cellulose derivatives including cellulose acetate, hydroxypropyl methylcellulose, hydroxypropyl cellulose, or combinations thereof.

6. The RNAi-nanoparticle conjugate of claim 1, wherein:
the ratio of the active ingredient to the polysaccharide is from 1:1 to about 1:5; and
the ratio of the nanoencapsulate to the RNAi silencer is from 25:0.1 to about 25:1.

7. A pharmaceutical composition comprising an RNAi-nanoparticle conjugate according to claim 1 and at least one pharmaceutically acceptable excipient.

8. The pharmaceutical composition of claim 7, wherein the pharmaceutical composition comprises from 1% to 99%, by weight RNAi-nanoparticle conjugate, based on the total weight of the pharmaceutical composition.

9. The pharmaceutical composition of claim 7, wherein the pharmaceutical composition comprises from 10% to 30%, by weight RNAi-nanoparticle conjugate, based on the total weight of the pharmaceutical composition.

10. The pharmaceutical composition of claim 7, wherein the pharmaceutical composition specifically suppresses an Ephb4 receptor.

11. The pharmaceutical composition of claim 10, wherein the Ephb4 receptor tyrosine kinase controls Wnt signaling.

12. A method for the synthesis of an RNAi-nanoparticle conjugate of claim 1, the method comprising:
(a) encapsulating the active ingredient in the polysaccharide to obtain a nanoencapsulate;
(b) fabricating the nanoencapsulate obtained in (a) into nanoparticles;
(c) immobilizing the nanoparticles obtained in (b) with the RNAi silencer at a temperature of from 40° C. to 60° C. to obtain a complex; and
(d) coating the complex obtained in (c) with the coating agent to obtain the RNAi-nanoparticle conjugate.

13. The method of claim 12, wherein the fabrication of the nanoencapsulate in (a) comprises electrospraying.

14. The method of claim 12, wherein the RNAi silencer comprises EphB4-shRNA and aids in the knockdown of target genes.

15. The method of claim 14, wherein the target genes are EphB4, β-catenin, and c-Myc.

16. The method of claim 12, wherein:
the RNAi-nanoparticle conjugate suppresses the expression of Ephb4 and c-Myc; and
the RNAi-nanoparticle conjugate relocalizes β-catenin from a nucleus to a cytoplasm.

17. A method of treating a cancer in a subject, the method comprising administering to the subject a therapeutically effective amount of the RNAi-nanoparticle conjugate of claim 1 or a pharmaceutical composition comprising the RNAi-nanoparticle conjugate and at least one pharmaceutically acceptable excipient.

18. The method of claim 17, wherein the cancer is breast cancer or colon cancer.

19. The method of claim 17, wherein the RNAi-nanoparticle conjugate or the pharmaceutical composition is administrated orally.
